(19) United States
(12) Patent Application Publication
(10) Pub. No.: US 2002/0187945 A1
(43) Pub. Date: Dec. 12, 2002

(54) Modulation of immune response by ribavirin

(76) Inventor: Robert Tam, Irvine, CA (US)

Correspondence Address: RUTAN & TUCKER, LLP, P.O. BOX 1950, COSTA MESA, CA, 92628-1950, US

(21) Appl. No.: 10/154,189

(22) Filed: May 20, 2002

Related U.S. Application Data
(63) Continuation-in-part of application No. 09/594,270, filed on Jun. 15, 2000.
Application No. 09/594,270 is a division of application No. 09/241,367, filed on Jan. 29, 1999, now abandoned.

(51) Int. Cl. A61K 31/7056; G06F 17/60

(52) U.S. Cl. 514/43; 705/2

(57) ABSTRACT

The response of an immune system to a challenge is modified by presenting the system with a nucleoside in a concentration selected to have an effect on a B7 marker that is inverse from the effect of the challenge. Contemplated challenges include allergens, neoplasm, virus, bacteria, infestation, and autoimmune reactions. Molecular markers of particular interest are B7-1 and B7-2. Preferred nucleosides are Ribavirin and Ribavirin analogs, especially provided within a concentration range between about 0.2 μM and about 5 μM, respectively, in a fluid containing cells expressing the B7 marker.

This application is a continuation-in-part of U.S. application Ser. No. 09/594,270, filed Jun. 15, 2000, which is division of a U.S. application Ser. No. 09/241,367, filed Jan. 21, 1999, now abandoned, and claims priority to U.S. Provisional Application No. 60/073,201, filed Jan. 30, 1998.

### FIELD OF THE INVENTION

The field of the invention is immunology.

### BACKGROUND OF THE INVENTION

In addition to the commonly employed physiological and phenotypical diagnostic parameters, diseases can sometimes be correlated with molecular markers such as polidy, mutations in specific genes, display of distinct cell surface markers and so forth. Many of these markers act as disease-specific predictors or indicators, and can thus be used as a diagnostic tool for a clearly defined physiological condition.

In recent years, many attempts have been made to correlate relatively complex diseases such as autoimmunity, asthma, cancer etc. with specific molecular markers. Several studies have found a direct or indirect involvement of the costimulatory molecules B7-1 and B7-2 in modulating the immune system in diseases. However, despite many detailed insights into the various expression levels of B7-1 and B7-2 in diseases obtained through such studies, a comprehensive and unified picture has not been elaborated (Hepatology 25, No.5, 1997, p1108-1114: Expression of costimulatory molecules B7-1 and B7-2 and human hepatocellular carcinoma; J. Cancer Res. Clin. Oncol. 124, No.7, 1998, p383-388: Expression of costimulatory molecules B7-1 and B7-2 on human gastric carcinoma; J. Neuroimmunol. 84, No.2, 1998, p179-187: Costimulatory CD80 (B7-1) and CD86 (B7-2) on cerebrospinal fluid cells in multiple sclerosis; J Neuroimmunol 91, No1-2, 1998, p198-203: B7-1 (CD80), B7-2 (CD86), interleukin-12 and transforming growth factor-beta mRNA expression in CSF and peripheral blood mononuclear cells from multiple sclerosis patients).

In many instances, apparently inconsistent correlations have been observed between B7-1, B7-2, and specific diseases (see FIG. 1). In some types of cancer, for example, B7-1 is present in relatively high amounts and B7-2 is present in relatively low amounts. In other types of cancers, B7-1 and B7-2 have exactly the opposite correlation (J. Cancer Res. Clin. Oncol. 124, No.7 1998, p383-388: Expression of costimulatory molecules B7-1 and B7-2 on human gastric carcinoma; Br. J. Haematol 102, No.5, 1998, p1257-1262: The expression of costimulatory molecules and their relationship to the prognosis of human acute myeloid leukemia: poor prognosis of B7-2-positive leukemia; Int. J. Mol. Med. 2, No.2, 1998 p167-171: Lack of B7-1 and B7-2 on head and neck cancer cells and possible significance for gene therapy).

B7-1 and B7-2 expression also show only inconsistent correlation with known cytokine patterns (see FIG. 2). For example, enhanced expression of B7-1 has been correlated with both up- and down-regulation of Type 1 responses, and B7-2 has also been correlated with both up- and down-regulation of Type 1 responses. The same can be said for the correlation with B7-1 and B7-2 with Type 2 response (see FIG. 1) (Am. J. Respir. Cell. Mol. Biol. 17, No.2, 1997, p235-242: Differential regulation of human, antigen-specific Type 1 and Type 2 responses by the B-7 homologues CD80 and CD86; J. Immunol. 156, No.8, 1996, p2387-2391: Costimulation of IL-4 production by murine B7-1 and B7-2 molecules).

Still further, it is not clear which drugs or even which drug categories would be effective in modulating B7-1 or B7-2 activity, and even if such drugs were identified, it remains unclear how to beneficially make use of these costimulatory molecules to modulate the immune system. Taking together all of these unknowns, there is still a considerable need to provide methods and compositions for modulating one or more of the B7 markers, especially as a means of affecting the response of an immune system to a given challenge.

### BRIEF DESCRIPTION OF THE DRAWINGS

FIG. 1 is a table correlating specific diseases and their correlation with B7-1 and B7-2 expression.

FIG. 2 is a table correlating various types of diseases with Type 1, Type 2, B7-1, and B7-2 expression.

FIG. 3 is a graphical representation of B7-1 and B7-2 expression in response to administration of Ribavirin in primed lymph node cells.

FIG. 4 is a graph showing in vivo modulation of contact hypersensitivity by Ribavirin.

FIG. 5 is an autoradiograph showing modulation of expression of B7-1 and B7-2 in contact hypersensitivity responses by Ribavirin.

FIG. 6 is a graphical representation of B7-1 and B7-2 expression in response to administration of Ribavirin in resting lymph node cells.

FIG. 7 is an autoradiograph showing modulation of expression of B7-1 and B7-2 in human peripheral blood mononuclear cells by Ribavirin.

### SUMMARY OF THE INVENTION

This invention provides methods and compositions by which the response of an immune system to a challenge is modified. In general, the response is modified by presenting the system with a nucleoside in a concentration selected to have an effect on a B7 marker that is inverse from the effect of the challenge.

In one aspect of preferred embodiments, the challenges are selected from the groups consisting of allergens, neoplasm, virus, bacteria, infestation, and autoimmune reaction. Molecular markers of particular interest are B7-1 and B7-2. In another aspect of preferred embodiments, the nucleoside is a Ribavirin analog, and in especially preferred embodiment, the nucleoside is Ribavirin. In yet another aspect of preferred embodiments, a sufficient nucleoside is provided to achieve a concentration range between about 0.2 μM and about 5 μM, respectively, in a fluid containing cells expressing the B7 marker.

In still another aspect of preferred embodiments, the challenge is correlated with an increase in Type 2 response, and application of the nucleoside is correlated with a decrease in Type 2 response.

### DETAILED DESCRIPTION OF SPECIFIC EMBODIMENTS

The present inventor has discovered that there is a surprising link between certain nucleosides, especially Ribavirin and its analogues, and expression of one or more of the B7 markers. Further discoveries revealed another unexpected link—that application of such nucleosides can be used to favorably affect the outcome of a disease or other challenge. In particular, a method of modulating a response of an immune system to a challenge has been discovered comprising: (a) correlating the challenge with an effect on a B7 marker; (b) correlating application of a nucleoside within a concentration range with modulation of the B7 molecular marker that is inverse to the effect; and (c) presenting the immune system with the nucleoside within the concentration range.

As used herein, the term “nucleoside” refers to a compound composed of any pentose or modified pentose moiety attached to a specific position of a heterocycle, to the natural position of a purine (9-position) pyrimidine (1-position), or to the equivalent position in an analog, including especially both D- and L-forms of nitrogenous bicyclic and monocyclic heterocycles. The term “D-nucleosides” refers to nucleoside compounds that have a D-ribose sugar moiety (e.g., Adenosine). The term “L-nucleosides” refers to nucleoside compounds that have an L-ribose sugar moiety. The term “nucleotide” means nucleosides in which phosphate esters are substituted on the 5′ position of a nucleoside.

The term “pharmaceutically acceptable salts” refers to any salt derived from inorganic and organic acids or bases.

The term “neoplasm” refers broadly to any sort of autonomous morbid growth of tissue that may or may not become malignant, including all manner of tumors and cancers.

The terms “treating” or “treatment” of a disease refer to executing a protocol, which may include administering one or more drugs to a patient, in an effort to alleviate signs or symptoms of the disease. Thus, “treating” or “treatment” do not require complete alleviation of signs or symptoms and do not require a cure, but specifically include protocols which have only a marginal effect (such as placebo effect) on the patient.

As used herein, the term “immune system” means any collection of immuno-competenT-cells that collectively identify and attack foreign entities, and that dynamically responds to new pathogens or other challenges. Examples of immune systems are human or other mammalian immune systems that include a spleen, thymus B-lymphocytes, T-lymphocytes and antibodies. An immune system as defined herein must have a cellular component, but may or may not have a humoral component. Where a humoral component is included in the immune system, the humoral component may include soluble molecules secreted from immuno-competenT-cells, including antibodies or interleukins. Examples for soluble molecules are IgG, IgM, IgE, IL2, IL4, or IL10.

Under this definition, whole blood, as well as blood depleted of fibrinogen, platelets, and erythrocytes is considered to comprise an immune system, since it contains immunocompetenT-cells that are capable of dynamically responding to new pathogens. Other immune systems are cell culture mediums containing immunocompetenT-cells. In contrast, a buffered solution of antibodies is not considered an immune system, since it does not contain a plurality of immunocompetenT-cells. In still other embodiments, human or other animals all contain immune systems as defined herein.

The term “challenge” is used herein to mean any component or event that provokes a response by the immune system. Challenges may be grouped in three categories: self, non-self, and altered self-challenges. Self-type challenges include cells or molecules, wherein the immune system and the challenge are from the same organism, self proteins or autologous proteins and fragments thereof. Examples include human blood cells, undifferentiated cells, antibodies or coagulation factors from the same human. Non-self-type challenges include cells, viruses, or molecules, wherein the immune system and the challenge are from different organisms, or the challenge is xenogenic. Examples include organs or cells from a non-identical donor, bacteria, viruses, or any type of molecules typical for other species, including endotoxins, enzymes, or structural proteins. Altered self-type challenges include cells or molecules wherein the immune system and the challenge are from the same organism, but wherein the challenge is subject to modifications and degradative or neoplastic changes. Examples of such modifications include modifying the profile of a B7 marker on antigen presenting cells. Examples of degradative changes include cells committed to apoptosis or necrotic tissue. Examples of neoplastic changes include induction of cancer.

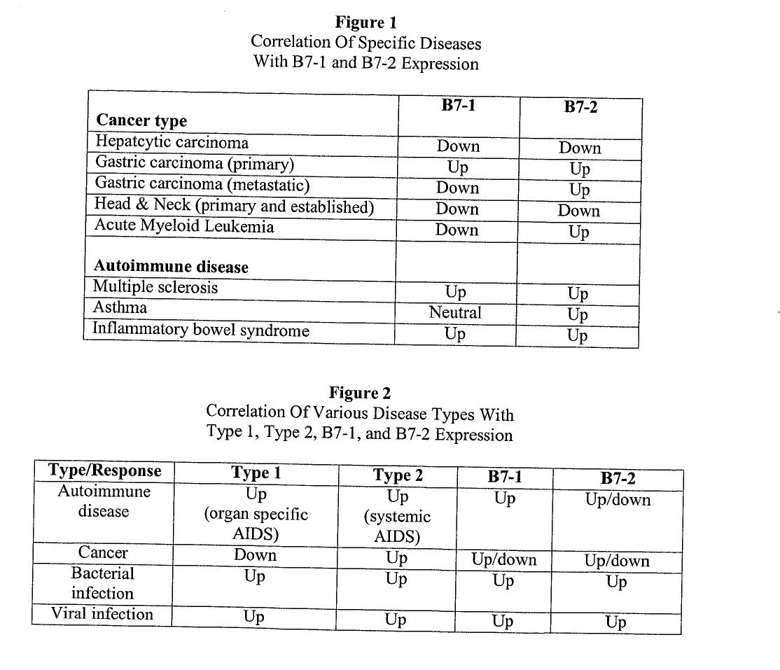

The terms “immune system response” and “immune response” are used herein to mean any response of an immune system to a challenge. Of particular interest in this application are immune responses that include modulation of a B7 marker. Such modulation may comprise any combination of increase or decrease in B7-1 and B7-2 expression. Thus, all of the responses tabulated in the tables of FIGS. 1 and 2 are examples of contemplated immune system responses.

Other contemplated immune system responses include engagement of cellular components in cell specific interactions or changes in genetic activity. Cell specific interactions may be cell-cell interactions or cell-challenge interactions. Examples for cell-cell interactions are T-cells contacting T-helper cells or T-helper cells contacting macrophages. Examples for cell-challenge interactions are antigen presenting cells incorporating the challenge, processing the challenge, and displaying the processed challenge on the cell surface, or B-cells displaying challenge specific antibodies on their cell surface and binding the challenge with the antibody. Changes in genetic activity may be rearrangements in genomic DNA, or selective activation of genes. Examples for rearrangements in genomic DNA are splicing events leading to affinity maturation of antibodies against the challenge or splicing events leading to a class switch between different classes of antibodies. Examples for selective activation of genes are increases or decreases of transcription or translation of genes coding for interleukins B7-1 or B7-2.

As used herein, producing a B7 effect that is ‘inverse” to the pattern associated with the challenge means that the B7 effect produced by the nucleoside alone is at least marginally in an opposite direction of that associated with the challenge alone. Thus, if the challenge is associated with reduced B7-1 expression, an inverse B7 effect would be one in which B7-1 is at least marginally elevated. Similarly, if the challenge is associated with an increased B7-2 expression, an inverse B7 effect would be one in which B7-2 is at least marginally reduced.

As used herein the term “presenting the immune system with a nucleoside” means that the nucleoside sufficiently contacts some component of the immune system to produce an immune system response. In preferred embodiments this means adding the nucleoside to a body. In other embodiments this means adding the nucleoside to a vessel, or other container of the immune system.

It should be appreciated that the definition of the term “presenting the immune system with a nucleoside” is sufficiently broad to include any combination of in-vivo, in-vitro, or ex-vivo contact. In-vivo may include injection, ingestion, transdermal delivery, or inhalation. Examples for various injections are intramuscular, intravenous, or subcutaneous injections. Examples for various forms of ingestion are tablets, syrups, or powders. Occlusive dressings, ointments or electrophoretic methods may achieve transdermal delivery. Inhalation may encompass methods of vaporizing or spraying.

In-vitro contact may be achieved by either dispensing a nucleoside containing solution to the immune system in a suitable vessel, or by dissolving the nucleoside in a solution that may or may not be part of the immune system. Examples for dispensing include automated or manual pipetting, dripping, pouring or injecting a nucleoside containing solution into the immune system. Alternatively, a nucleoside may also be dissolved in a fluid by stirring, mixing or pouring Ribavirin in the fluid. This fluid may comprise the immune system or may be a carrier solution including a buffer, isotonic solutions, or blood. This carrier may then be dispensed to the immune system.

Ex-vivo contact may be achieved in several steps comprising (1) collecting part of the immune system from a source, (2) administering the nucleoside to the immune system, and (3) returning the immune system at least in part to the source. Collecting part of the immune system may be done by retrieving part of the immune system from an in-vivo or in-vitro source. Examples of in-vivo sources are vertebrate animals, including humans, and invertebrate animals. Retrieving may be done by venipuncture, eye bleeds, or pinpricks. Examples of in-vitro sources are cell cultures containing the immune system, treated or stored blood. The retrieving may be done by any means of fluid transfer, for example automated or manual pipetting, aspiration, dripping and so on. Returning the immune system to the source may be done by any means of fluid transfer. This may be in the case of an in-vitro source automated or manual pipetting, aspiration, dripping or in the case of an in-vivo source injecting intravenously.

Contemplated nucleosides are Ribavirin (1-β-D-Ribofuranosyl-1,2,4-Triazole-3-Carboxamide), and analogs thereof. To clarify matters, the term “Ribavirin analogs” means any derivatized Ribavirin in which (1) one or more of the hydroxyl groups is substituted by a non-hydroxyl moiety having less than 25 atoms, including H, lower alkyl, lower aryl, lower aralkyl, lower alkyl alkenyl, halogen, and so forth, and independently one or more of the hydrogens is substituted by a non-hydrogen moiety having less than 25 atoms, including OH, lower alkyl, lower aryl, lower aralkyl, lower alkyl alkenyl, halogen, and so forth.

The ribavirin, ribavirin analog, or other nucleoside is preferably formulated in a buffered aqueous solution. In alternative embodiments, however, the nucleoside may be formulated in many other liquid or solid forms. Liquid forms may be solutions comprising pure solvents including water, DMSO or ethanol. Liquid forms may also comprise solutions having mixtures of solvent with other solvents, or dissolved solids including water-ethanol mixtures, water-DMSO mixtures, and buffers. Furthermore, liquid forms of nucleosides may be mixed, for example, with consistency-modifying substances to form gels, creams or ointments. Examples are amphiphilic molecules, waxes or gelatin. Solid forms may comprise solids that may or may not be active ingredients. Examples for active ingredients are buffers, ion-exchange resins including MOPS, phosphates or citrates. Examples of inactive ingredients include starch, cellulose or silica. Furthermore, solid forms may be in various preparations, including tablets, capsules, powder etc.

In preferred embodiments, a sufficient nucleoside is provided to achieve a concentration range between about 0.2 μM and about 5 μM, respectively, in a fluid containing cells expressing the B7 marker. Less preferred embodiments contemplate other concentrations within the range of 0.1 μM to about 10 μM.

In another aspect of preferred embodiments, the challenge is correlated with an increase in Type 2 response and application of the nucleoside is correlated with a decrease in Type 2 response. Type 2 responses can be understood as follows:

Mammalian immune systems contain two major classes of lymphocytes: B lymphocytes (B cells), which originate in the bone marrow and T lymphocytes (T-cells) which originate in the thymus. B cells are largely responsible for humoral immunity (i.e., antibody production), while T-cells are largely responsible for cell-mediated immunity. T-cells are generally considered to fall into two subclasses, helper T-cells and cytotoxic T-cells. Helper T-cells activate other lymphocytes, including B cells and cytotoxic T-cells, and macrophages, by releasing soluble protein mediators called cytokines that are involved in cell-mediated immunity. As used herein, lymphokines are a subset of cytokines.

Helper T-cells are also generally considered to fall into two subclasses, Type 1 and Type 2. Type 1 cells (also known as Th1 cells) produce interleukin 2 (IL-2), tumor necrosis factor (TNFα) and interferon gamma (IFNγ), and are responsible primarily for cell-mediated immunity such as delayed type hypersensitivity and antiviral immunity. In contrast, Type 2 cells (also known as Th2 cells) produce interleukins, IL4, IL-5, IL-6, IL-9, IL-10 and IL-13, and are primarily involved in assisting humoral immune responses such as those seen in response to allergens, e.g. IgE and 1gG4 antibody isotype switching (Mosmann, 1989, Annu Rev Immunol, 7:145-173).

As used herein, the terms “Type 1 responses” and “Type 2 responses” are meant to include the entire range of effects resulting from induction of Type 1 and Type 2 lymphocytes, respectively. Among other things, such responses include variation in production of the corresponding cytokines through transcription, translation, secretion and possibly other mechanisms, increased proliferation of the corresponding lymphocytes, and other effects associated with increased production of cytokines, including motility effects.

Either Type 1 or Type 2 responses can be selectively suppressed while the other is either induced or left relatively unaffected, and either of Type 1 or Type 2 responses can be selectively induced while the other is either suppressed or left relatively unaffected. Certain nucleosides such as ribavirin are effective in selectively modulating Type 1 and Type 2 responses relative to one another. Determination of which nucleosides are effective in reducing Type 2 response is readily determined by experimentation.

It is contemplated that the methods described herein may be used to treat a wide variety of diseases, and in fact any disease which responds favorably to such treatment. Among other things it is specifically contemplated that such combinations may be used to treat an allergen (allergy), a neoplasm (cancer), a virus (viral infection), a bacterium (bacterial infection), an infestation, or an autoimmune disease.

Infections contemplated to be treated with the nucleosides of the present invention include respiratory syncytial virus (RSV), hepatitis B virus (HBV), hepatitis C virus (HCV), herpes simplex type 1 and 2, herpes genitalis, herpes keratitis, herpes encephalitis, herpes zoster, human immunodeficiency virus (HIV), influenza A virus, hantann virus (hemorrhagic fever), human papilloma virus (HPV), measles, and fungus. It is especially contemplated that combinations claimed herein will be useful in treating chronic viral and bacterial infections, including HIV, Tuberculosis, leprosy and so forth.

Infestations contemplated to be treated with the nucleosides of the present invention include intracellular protozoan infestations, as well as helminth and other parasitic infestations. Again, it is especially contemplated that combinations claimed herein will be useful in treating chronic infestations. inhibiting the spread of viruses from transformed cells to other normal cells and/or arresting the growth of virus-transformed cells.

Allergies contemplated to be treated include all IgE and IgG allergies, hyper IgE syndrome, and dermatic conditions such as atopic dermatitis. It is also contemplated that the claimed methods can be used to treat transplant rejection, (graft vs. host disease) and implant reactions.

Autoimmune diseases can be classified as either non-organ-specific or organ-specific. Non-organ-specific autoimmune diseases include rheumatoid arthritis, gout and gouty arthritis, Systemic Lupus Erythematosus (SLE), Sjogren syndrome, scleroderma, polymyositis and dermomyositis, ankylosing spondylitis, and rheumatic fever. Organ-specific autoimmune diseases are known for virtually every organ, including insulin-dependent diabetes, thyroid diseases (Graves disease and Hashimoto thyroiditis), Addison disease, and some kidney and lung diseases including allergy, asthma, multiple sclerosis, myasthenia gravis, uveitis, psoriasis, forms of hepatitis and cirrhosis, celiac disease, inflammatory bowel disease, and some types of male and female infertility. Autoimmune processes may also be stimulated by viral infections including the HIV virus, which may result from rejection of transplantation, and may accompany certain tumors, or be precipitated by exposure to some chemicals.

Synthesis

Synthesis of ribavirin is well known, and synthesis of ribavirin analogs is contemplated.

Administration

It is contemplated that nucleosides according to the present invention will be administered in any appropriate pharmaceutical formulation, and under any appropriate protocol. Preferred dosages and protocols are contemplated to be best established through experimentation with particular patients. Such experimentation need not be extensive, and it is contemplated that nucleosides will be administered in humans at between about 100 mg/day and about 5,000 mg/day. In humans and other systems, the ribavirin or other nucleoside is especially contemplated to be provided under parameters that produce a concentration of the nucleoside in a fluid containing cells expressing a B7 marker between about 0.2 μM and about 5 μM, respectively.

Of course, where treatment of a disease is concerned, one of ordinary skill in the art will recognize that a therapeutically effective amount will vary with the infection or condition to be treated, its severity, the treatment regimen to be employed, the pharmacokinetics of the agent used, as well as the patient (animal or human) treated. Thus, effective dosages may range from 1 mg/kg of body weight, or less, to 25 mg/kg of body weight or more. In general, a therapeutically effective amount of the “second” drug is contemplated to range from slightly less than about 1 mg./kg. to about 25 mg./kg. of the patient, depending upon the nucleoside used, the condition or infection treated, and the route of administration. This dosage range generally produces effective blood level concentrations of active nucleoside ranging from about 0.04 to about 100 micrograms/cc of blood in the patient. It is contemplated, however, that appropriate patient-specific regimens will be developed by administering a small amount, and then increasing the amount until either the side effects become unduly adverse, or the intended effect is achieved.

Administration of nucleosides according to the present invention may take place orally, parenterally (including subcutaneous injections, intravenous, intramuscularly, by intrastemal injection or infusion techniques), by inhalation spray, rectally, or topically and so forth, and in dosage unit formulations containing conventional non-toxic pharmaceutically acceptable carriers, adjuvants and vehicles.

It is contemplated that nucleosides according to the present invention can be formulated in admixture with a pharmaceutically acceptable carrier. For example, the nucleosides of the present invention can be administered orally as pharmacologically acceptable salts. Because the nucleosides of the present invention are mostly water soluble, they can be administered intravenously in physiological saline solution (e.g., buffered to a pH of about 7.2 to 7.5). Conventional buffers such as phosphates, soluble, they can be administered intravenously in physiological saline solution (e.g., buffered to a pH of about 7.2 to 7.5). Conventional buffers such as phosphates, bicarbonates or citrates can be used for this purpose. Of course, one of ordinary skill in the art may modify the formulations within the teachings of the specification to provide numerous formulations for a particular route of administration without rendering the compositions of the present invention unstable or compromising their therapeutic activity. In particular, the modification of the present nucleosides to render them more soluble in water or another vehicle, for example, may be easily accomplished by minor modifications (salt formulation, esterification, etc.), which are well within the ordinary skill in the art. It is also well within the ordinary skill of the art to modify the route of administration and dosage regimen of particular nucleosides in order to manage the pharmacokinetics of the contemplated nucleosides for maximum beneficial effect in patients.

In certain pharmaceutical dosage forms, the pro-drug form of administered nucleosides, especially acylated (acetylated or other) derivatives, pyridine esters and various salt forms of the present nucleosides are preferred. One of ordinary skill in the art will recognize how to readily modify the present nucleosides to pro-drug forms to facilitate delivery of active nucleosides to a target site within the host organism or patient. One of ordinary skill in the art will also take advantage of favorable pharmacokinetic parameters of the pro-drug forms, where applicable, in delivering the contemplated nucleosides to a targeted site within the host organism or patient to maximize the intended effect of the nucleoside.

In addition, contemplated nucleosides may be administered separately or together, and when administered separately this may occur in any order. The amounts of the active ingredient(s) pharmaceutically active agent(s) and the relative timings of administration will be selected in order to achieve a desired combined therapeutic effect.

Administration routes of contemplated nucleosides may range from continuous (intravenous drip) to several oral administrations per day (for example, Q.I.D.) and may include oral, topical, parenteral, intramuscular, intravenous, sub-cutaneous, transdermal (which may include a penetration enhancement agent), buccal and suppository administration, among other routes of administration.

In treatments according to the present invention, a therapeutically effective amount of a nucleoside is preferably intimately admixed with a pharmaceutically acceptable carrier according to conventional pharmaceutical compounding techniques to produce a dose. A carrier may take a wide variety of forms depending on the form of preparation desired for administration, e.g., oral or parenteral. In preparing pharmaceutical compositions in oral dosage form, any of the usual pharmaceutical media may be used. Thus, for liquid oral preparations such as suspensions, elixirs and solutions, suitable carriers and additives including water, glycols, oils, alcohols, flavoring agents, preservatives, coloring agents and the like may be used. For solid oral preparations such as powders, tablets, capsules, and for solid preparations such as suppositories, suitable carriers and additives including starches, sugar carriers, such as dextrose, mannitol, lactose and related carriers, diluents, granulating agents, lubricants, binders, disintegrating agents and the like may be used. If desired, the tablets or capsules may be enteric-coated or prepared for sustained release by standard techniques.

For parenteral formulations, the carrier will usually comprise sterile water or aqueous sodium chloride solution, though other ingredients including those that aid dispersion may be included. Of course, where sterile water is to be used and maintained as sterile, the compositions and carriers must also be sterilized. Injectable suspensions may also be prepared, in which case appropriate liquid carriers, suspending agents and the like may be employed.

It will also be appreciated that in general, the most preferred uses according to the present invention are those in which the active nucleosides are relatively less cytotoxic to the non-target hosT-cells and relatively more active against the target. In this respect, it may also be advantageous that L-nucleosides may have increased stability over D-nucleosides, which could lead to better pharmacokinetics. This result may be attained because L-nucleosides may not be recognized by enzymes, and therefore may have longer half-lives.

### EXAMPLES

It has previously been recognized that the costimulatory molecules B7-1 (CD80) and B7-2 (CD86), through their engagement with CD28 on T-cells can provide the second signal required to trigger MHC-restricted T cell activation (see e.g., J. Biol. Chem. 1995 Sep 8;270(36):21181-7). Furthermore, it has been demonstrated that B7-1 and B7-2 are expressed on professional APCs such as dendritic cells, monocytes, macrophages and activated B cells as well as in activated T-cells (see e.g., J Exp Med 1993 Mar 1;177(3):845-50).

Moreover, various groups have reported that cytotoxic T-lymphocyte (CTL) response is triggered by B7 costimulatory molecules (see e.g., J Immunol. 1995 Jan 1;154(1):97-105). Furthermore, it is also well known that an infection with various viruses, specifically infection with HCV typically results in an impaired CTL response (see e.g., Springer Semin Immunopathol 1997;19(1):57-68, or Hepatology 1997 Mar;25(3):705-12). Recent reports have shown that introduction of viral antigenic determinants, for example the surface antigen of the hepatitis B virus, coexpressed with B7-1 or B7-2 molecules can lead to virus-specific cell-mediated immunity.

Consequently, the inventors contemplate that modulation of expression of B7 molecules may play an essential role in overcoming a challenge presented to the immune system, and particularly in generating antiviral immunity. More particularly, based on previous observations and further experiments (not shown herein) the inventors contemplate that challenges (especially viral infections) associated with a change of B7 expression in one direction (i.e., up or down) may be treated, if not cured, by administration of a drug that modulates the changed B7 expression in the opposite direction.

Consequently, the following experiments were conducted to determine whether ribavirin, ribavirin prodrugs and analogs, or other nucleoside analogs may be effective to treat various challenges via enhancement of the costimulatory B7/CD28 pathway.

### B7 Expression Following Ribavirin Treatment in Lymph Node Cells from Ag-Primed BALB/c and C57BL/6 Mice

The effect of ribavirin on B7-1 expression and B7-2 expression, at the time of challenge, in lymph node cells isolated from Ag-primed BALBc and C57BL/6 mice was examined. Mice were primed epicutaneously with dinitrofluorobenzene (DNFB) 6 days prior to concomitant ribavirin treatment (5 μM) and in vitro challenge using an immobilized anti-αβ TCR antibody. Unexpectedly, we observed opposing effects of ribavirin on DNFB-induced B7-1 expression and B7-2 expression in these two mouse strains.

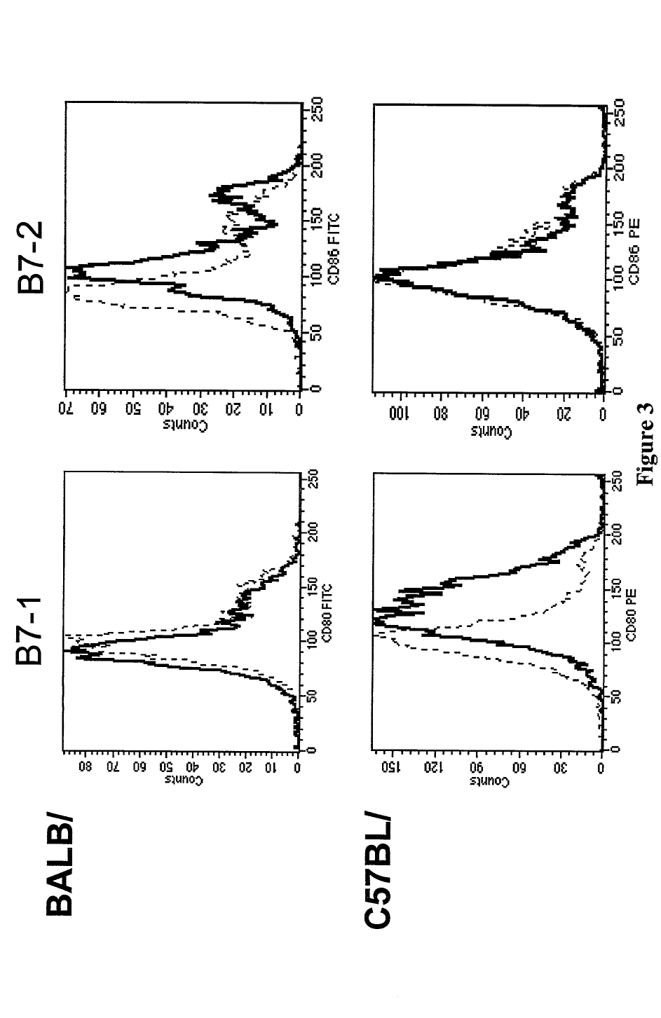

FIG. 3 shows that in BALB/c lymph node cells (top panel), ribavirin (RIB) enhanced B7-2 expression but not B7-1 expression. In contrast, in C57BL/6 mice (bottom panel), ribavirin enhanced B7-1 expression but not B7-2 expression (B7-1 levels and B7-2 levels were determined following FACS analysis using fluorescently labeled B7-1 antibodies and B7-2 antibodies (Ab) and are expressed as mean channel fluorescence).

### Contact Hypersensitivity (CHS) Responses in Ribavirin-Treated BALB/c and C57BL/6 Mice

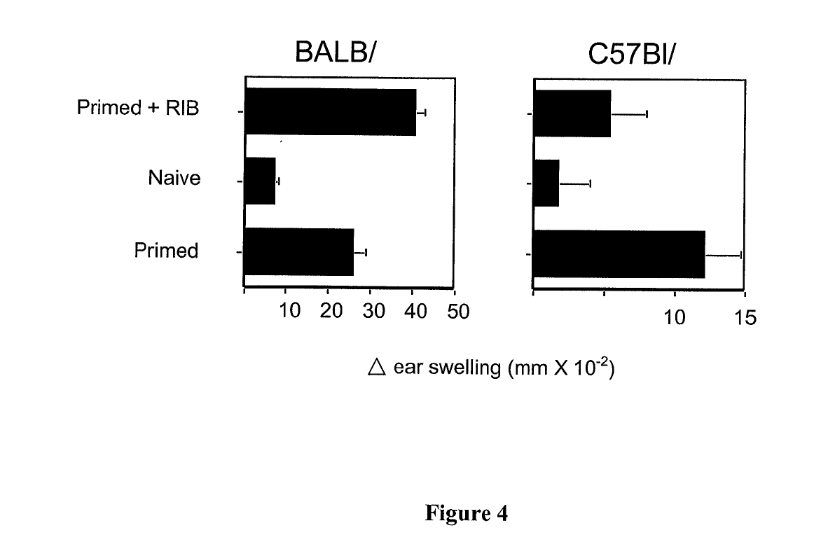

The functional effect of this differential effect on B7-1 and B7-2 mediated by RIB was examined in an in vivo murine model of contact hypersensitivity, and a B7-2 dependent immune response (Eur. J. Immunol, 1996. 26: 880-885). Briefly, animals were primed with 2,4-dinitro-1-fluorobenzene (DNFB) (0.3%) epicutaneously on the abdomen and challenged 6 days later on each ear with 0.12% DNFB. Ribavirin-treated animals were injected intraperitoneally at the time of challenge with an optimal dose of 0.5 mg/kg in 50 μl PBS. Ear measurements were taken before challenge and 24 hours after challenge. Response to DNFB was expressed as the difference between the two ear measurements. FIG. 4 shows the inflammatory ear responses in BALB/c and C57BL/6 mice with or without ribavirin injection. Ear responses in BALB/c mice were augmented following ribavirin (RIB) treatment but were impaired in ribavirin-treated C57BL/6 mice.

### B7-1 and B7-2 Expression in Lymph Nodes from Ribavirin-Treated BALB/c and C57BL/6 Mice

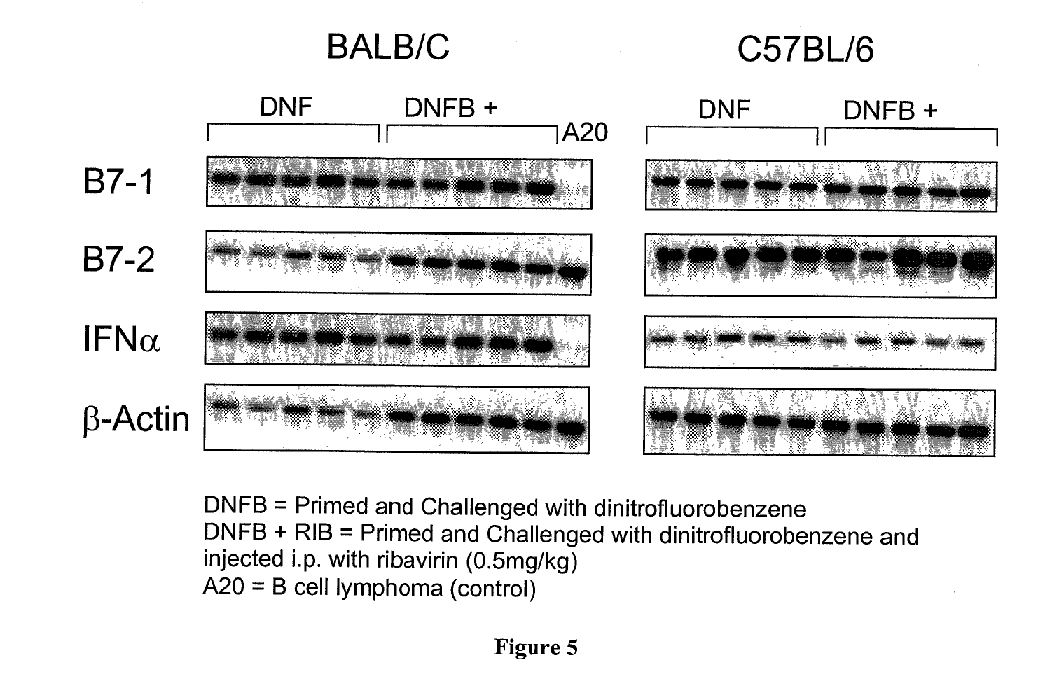

To determine the effect of ribavirin on the functional role of B7-1 and B7-2 on contact hypersensitivity (CHS) responses, we measured B7-1 and B7-2 mRNA levels in RNA isolated from the lymph node cells of the animals used in the above CHS experiments. Following RT-PCR with specific primers, electrophoresis and Southern blotting, PCR products were analyzed by hybridization with 32P-labeled specific probes. FIG. 5 shows that in BALB/c mice, B7-2 mRNA expression in lymph node cells was augmented following ribavirin-treatment, but B7-1 was unaffected. In contrast, in C57BL/6 mice, B7-1 mRNA expression was enhanced by ribavirin, but B7-2 mRNA expression was unaffected. This data shows that ribavirin-mediated increase in CHS responses was associated with an enhancement of B7-2 in BALB/c mice whereas in C57BL/6 mice ribavirin decreased CHS responses, and this was associated with an enhancement of B7-1 expression.

### B7-1 and B7-2 Expression in Splenocytes from BALB/c and C57BL/6 Mice Following Treatment with Ribavirin and IFNα

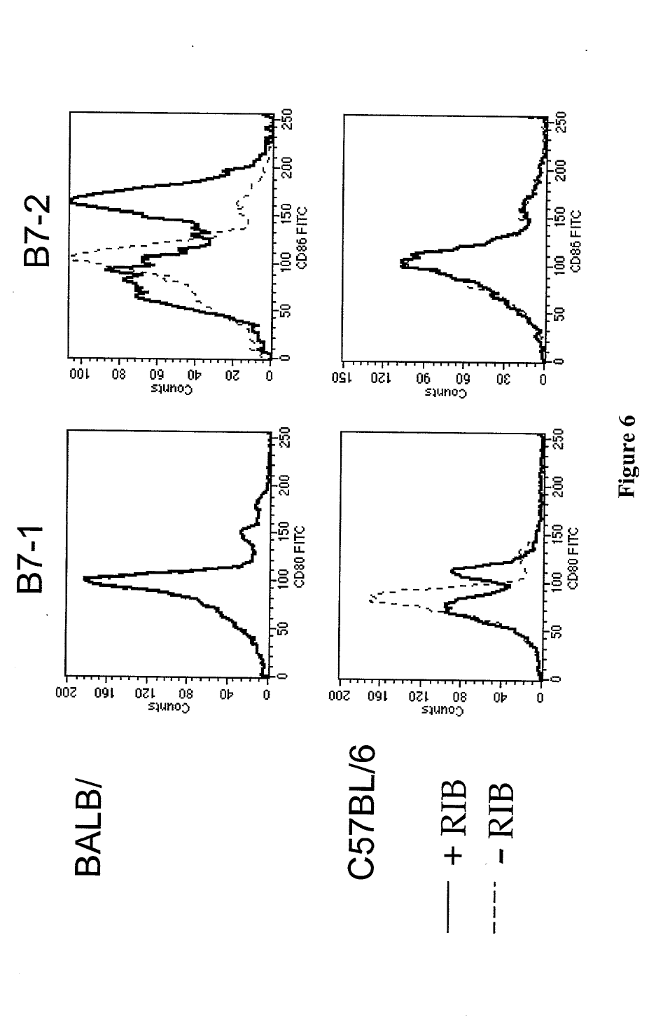

We determined the levels of B37-1 and B7-2 by FACS analysis from IFNα-treated splenocytes from unstimulated BALB/c and C57B6 mice in order to determine whether the effect of ribavirin on the functional role of B7-1 and B37-2 was a) inducible in resting, unstimulated lymphocytes, b) required the action of a B7 inducer such as IFNα, or c) elicited the same affects on the two mouse strains upon replacing antigen stimulation with a B7 inducer. FIG. 6 shows that in BALB/c splenocytes (top panel), IFNα-induced B7-2 expression but not B7-1 expression and was augmented following ribavirin-treatment. In contrast, in C57BL/6 splenocytes (bottom panel), IFNα-induced B7-1 expression was enhanced by ribavirin but B7-2 expression was unaffected. This data shows that ribavirin mediated similar effects on B7-1 and B7-2 expression after substitution of antigen stimulation with a B7 inducer such as the cytokine IFNα. Similar effects were not seen with RIB without IFNα.

### B7-1 and B7-2 Expression in Human Peripheral Blood Mononuclear Cells Following Treatment with RIB and the B7 Inducing Cytokines IFNα, IFNβ and TNFα

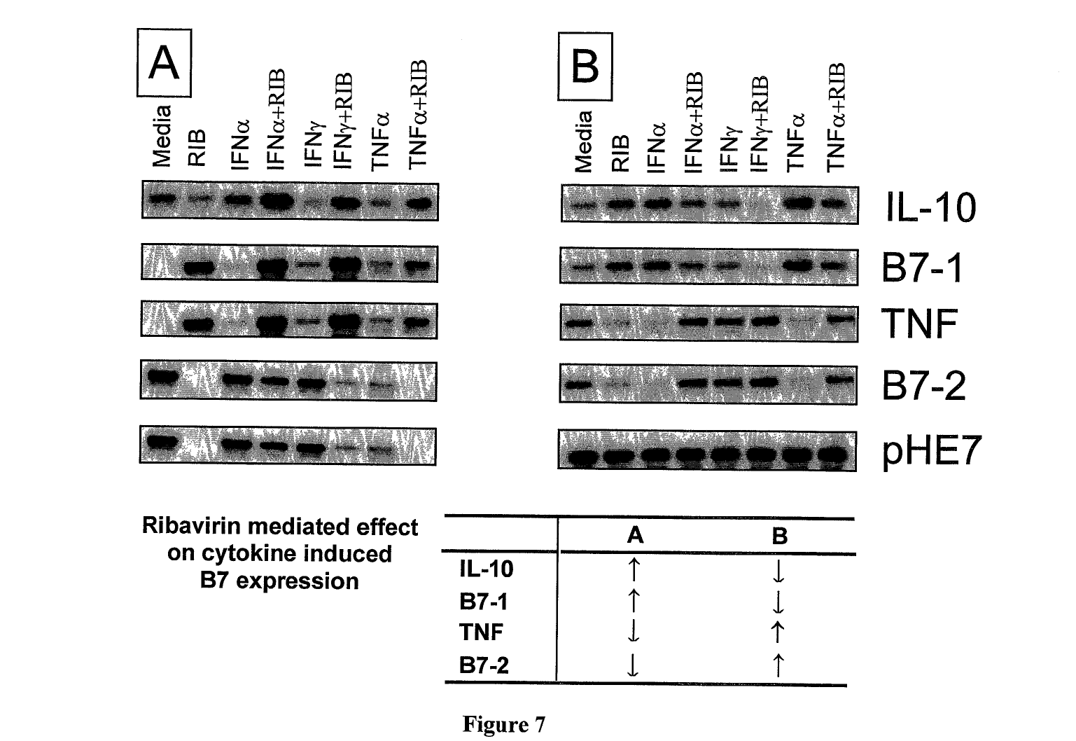

The differential effect by ribavirin on B7-1 and B7-2 expression as observed in the two mouse strains was examined in human peripheral blood mononuclear cells (PBMCs). Here we used three cytokines, IFNα, IFNβ, and TNFα to induce B7-1 and B7-2 expression. FIG. 7 shows the effect of a 24 hour incubation of these B7-inducing cytokines with PBMCs in the presence or absence of ribavirin. Two distinct outcomes were observed. In the left panel (A), B7-1 mRNA expression (as determined by RT-PCR) is increased upon addition of ribavirin whereas B7-2 expression is decreased, both observations correlating with a concomitant increase in IL-10 and a concomitant decrease in TNFα for all three B7-inducing cytokines. Conversely, in the right hand panel (B), ribavirin increased B7-2 expression and decreased B7-1 expression whilst IL-10 levels dropped and TNFα levels increased.

Therefore, it should be appreciated that the nucleoside analog Ribavirin is effective to enhance expression of both B7-1 and B7-2 costimulatory molecules. Furthermore, it should be recognized that differential enhancement of B7 expression can be achieved following antigenic stimulation (e.g., in two distinct strains of mice: in BALB/c mice B7-2 is increased, which led to an increase in contact hypersensitivity and (CHS) responses to dinitrofluorobenzene, whereas in C57BL/6 mice, B7-1 levels are increased, which was associated with impaired CHS responses) and without antigenic stimulation (e.g., in cells treated with IFN-alpha). Furthermore, it should be appreciated that in human PBMCs, differential enhancement of cytokine-induced B7-1 and B7-2 expression can be achieved (here, cross-modulation of TNFα and IL-10 resulted in opposing ribavirin-mediated effects on B7-1 and B7-2).

Consequently, it is contemplated that Ribavirin, and various other nucleoside analogs may be employed to treat a disease by modulating B7-1 and/or B7-2 expression. Particularly contemplated aspects of treatment include treatment of diseases which are associated with a change in B7-1 and/or B7-2 expression, or diseases in which an increase or decrease in B7-1 and/or B7-2 expression is beneficial.

For example, where an enhanced immune response against an antigen is desired, Ribavirin may be administered at a dosage effective to increase expression of B7-1 and/or B7-2 expression, thereby providing an enhanced CTL response against the antigen. Especially contemplated antigens are viral and bacterial antigens, which may or may not be associated with the corresponding virus or bacterium. Consequently, contemplated methods may be useful in treatment of infectious diseases and/or in providing protective immunity (i.e., vaccination).

Examples for an enhanced immune response include an increase in antibody titer against an antigen, switch of antibody class(es), increase in antigen-presenting cells, and an increased CTL-response, all of which can be readily determined by a person of ordinary skill in the art (see e.g., Advanced Methods in Cellular Immunology by R. Fernandez-Botran and V. Vetvicka; CRC Press; ISBN: 0849321255, or in Antibody Techniques by Vedpal S. Malik and Erik P. Lillehoj; Academic Press; ISBN: 0124664601). Furthermore, enhanced immune response may also be determined by reduction of the antigen in the organism, and numerous methods for determination the concentration of antigens are well known in the art (ELISA, bacterial and viral cultures, etc.). On a systemic level, an enhanced immune response may be determined by reduction in disease symptoms associated with the presence of the antigen (or antigen-associated host).

In another example, where an exacerbated immune response (e.g., autoimmune disease or contact hypersensitivity) against an antigen (self or non-self) is present in an organism, it is contemplated that administration of Ribavirin may be administered at a dosage effective to decrease expression of B7-1 and/or B7-2 expression, thereby providing a reduced (auto)immune response against the antigen.

Examples for an exacerbated immune response particularly include an increased antibody titer against an antigen, an increased CTL response towards the antigen, and consequently, a reduced (auto)immune response against the antigen may be readily determined by a person of ordinary skill in the art in a manner similar to those described above.

Consequently, it is contemplated that a method of changing expression of a B7 marker on a lymphocyte will comprise a step of presenting Ribavirin to the lymphocyte at a concentration effective to change the expression. In particularly contemplated aspects, the lymphocyte is in an environment that has a reduced cytotoxic T-cell response against an antigen. Thus, preferred B7 markers are B7-1 or B7-2, wherein the change in expression is an increase in B7-1 or B7-2, which advantageously will increase the cytotoxic T-cell response against the antigen. Consequently, it is contemplated that the increased cytotoxic T-cell response will reduce the concentration of an antigen (e.g., bacterial or viral antigen (hepatitis virus, and especially HCV)).

It is further contemplated that preferred methods may also be advantageous where the lymphocyte is in an environment that has an exacerbated immune response against an antigen (e.g., contact hypersensitivity reaction or autoimmune reaction). Therefore, it is contemplated that the B7 marker is B7-1 or B7-2, and wherein the change in expression is a decrease in B7-1 or B7-2. In particularly preferred aspects, the concentration of Ribavirin is effective to decrease expression in B7-1 or B7-2, thereby decreasing the exacerbated immune response against the antigen.

Therefore, it should be particularly recognized that administration of Ribavirin to a mammal, and especially to a human has numerous beneficial effects. More specifically, in addition to the well-known immunomodulatory effect via Th1/Th2 modulation and direct antiviral effect against selected viruses, administration of Ribavirin in a dosage range between about 0.1 to 100 mg/kg (and more typically between 1 to 20 mg/kg) will result in a modulation of B7-1 and/or B7-2 expression. For example, stimulation of B7-1 and/or B7-2 expression is particularly advantageous in patients with reduced CTL response since an increased B7-1 and/or B7-2 expression is believed to significantly enhance a CTL response.

Consequently, the inventors contemplate that recognition of particular additional beneficial effects of Ribavirin will significantly increase the value of Ribavirin as a product. Thus, a method of selling a product containing Ribavirin may include a step in which information is received that Ribavirin may change expression of a B7 marker on a lymphocyte. In a further step, the product containing Ribavirin is sold to a buyer.

In especially preferred aspects of contemplated methods, the lymphocyte is in an environment that has a reduced cytotoxic T-cell response against an antigen, and the B7 marker is B7-1 or B7-2, wherein the change in expression is an increase in B7-1 or B7-2. Therefore, it should be recognized that Ribavirin is effective at a physiologically acceptable concentration to increase expression in B7-1 or B7-2, thereby increasing the cytotoxic T-cell response against the antigen, wherein the cytotoxic T-cell response will reduce the concentration of an antigen (e.g., bacterial or viral antigen, and especially an antigen of a hepatitis virus such as HCV).

It is further contemplated that the lymphocyte may also be in an environment that has an exacerbated immune response against an antigen, an exemplary antigen in such environments may elicit a contact hypersensitivity reaction. Alternatively, contemplated antigens may also comprise a self-antigen that elicits an autoimmune reaction. Thus, it is also contemplated that the B7 marker is B7-1 or B7-2, wherein the change in expression is a decrease in B7-1 or B7-2. Consequently, it is contemplated that the concentration of Ribavirin is effective to decrease expression in B7-1 or B7-2, thereby decreasing the exacerbated immune response against the antigen. In yet further contemplated aspects, the change in expression in the B7 marker is achieved using administration of the Ribavirin to a person at dosage range between about 0.1 mg/kg and 100 mg/kg.

Contemplated sellers include manufacturers and non-manufacturers of Ribavirin, and therefore include fine-chemical merchants, pharmacists, and drug manufacturers. It is further contemplated that the step of receiving information may be accomplished by various means, and especially preferred means include reading of the information in a journal, patent, patent application, or letter, as well as experimental data or graphical representations thereof.

Thus, methods have been disclosed that employ ribavirin or other nucleosides to advantageously modulate a B7 molecular marker. While specific embodiments have been disclosed herein, the scope of the invention is not be limited except through interpretation of the appended claims.

What is claimed is:

1. A method of selling a product containing Ribavirin, comprising:
receiving information that the Ribavirin may change expression of a B7 marker on a lymphocyte;
selling the product containing the Ribavirin to a buyer.

2. The method of claim 1 wherein the lymphocyte is in an environment that has a reduced cytotoxic T-cell response against an antigen.

3. The method of claim 2 wherein the B7 marker is B7-1 or B7-2, and wherein the change in expression is an increase in B7-1 or B7-2.

4. The method of claim 3 wherein the Ribavirin is effective at a physiologically acceptable concentration to increase expression in B7-1 or B7-2, thereby increasing the cytotoxic T-cell response against the antigen.

5. The method of claim 4 wherein the cytotoxic T-cell response reduces a concentration of an antigen.

6. The method of claim 5 wherein the antigen is a bacterial or viral antigen.

7. The method of claim 6 wherein the viral antigen is an antigen of a hepatitis virus.

8. The method of claim 7 wherein the hepatitis virus is a hepatitis C virus.

9. The method of claim 1 wherein the lymphocyte is in an environment that has an exacerbated immune response against an antigen.

10. The method of claim 9 wherein the antigen elicits a contact hypersensitivity reaction.

11. The method of claim 9 wherein the antigen comprises a self-antigen that elicits an autoimmune reaction.

12. The method of claim 9 wherein the B7 marker is B7-1 or B7-2, and wherein the change in expression is a decrease in B7-1 or B7-2.

13. The method of claim 12 wherein the concentration of the Ribavirin is effective to decrease expression in B7-1 or B7-2, thereby decreasing the exacerbated immune response against the antigen.

14. The method of claim 1 wherein the change in expression is achieved using administration of Ribavirin to a person at a dosage range between about 0.1 mg/kg and 100 mg/kg.
